(19) United States
(12) Patent Application Publication
(10) Pub. No.: US 2020/0121385 A1
(43) Pub. Date: Apr. 23, 2020

(54) TREATMENT SYSTEM

(71) Applicant: Olympus Corporation, Tokyo (JP)

(72) Inventor: Masato Narisawa, Tokyo (JP)

(73) Assignee: Olympus Corporation, Tokyo (JP)

(21) Appl. No.: 16/661,019

(22) Filed: Oct. 23, 2019

Related U.S. Application Data
(63) Continuation of application No. PCT/JP2017/016423, filed on Apr. 25, 2017.

(51) Int. Cl. A61B 18/14; A61B 18/08; A61B 18/10

(52) U.S. Cl. CPC A61B 18/1445; A61B 2018/00708; A61B 18/10; A61B 18/085

(57) ABSTRACT

The disclosed technology is directed to a treatment system having a control device and a treatment instrument configured to be attached to the control device. The treatment instrument includes an elongated heat generating structure element having a heat transfer member and a plurality of heat-generating portions coupled to one another. The heat transfer member is configured to transmit thermal energy to the treatment target. A power source portion supplies electrical power to the plurality of heat-generating portions. A switch portion selects one target heat-generating portion from the plurality of heat-generating portions to be used as a target to which electrical power is to be supplied from the power source portion. A switch control portion is used to control operation of the switch portion and an energization control portion is used to control at least one of a switching timing of the target heat-generating portion.

### CROSS-REFERENCE TO RELATED APPLICATION

This application is a continuation application of PCT Application No. PCT/JP 2017/016423 filed on Apr. 25, 2017, which is hereby incorporated by reference in its entirety.

### TECHNICAL FIELD

The disclosed technology relates generally to a treatment system, and more particularly, some embodiments relate to a treatment system for use with a treatment instrument that can appropriately treat a treatment target such as biological tissue.

### DESCRIPTION OF THE RELATED ART

Known treatment systems include those which have a grasping portion. The grasping portion includes a heat-generating portion, which generates heat upon energization, and is used to grasp a treatment target, for example, a biological tissue, such that the biological tissue is subjected to treatment, specifically to joining or anastomosis, resection, or the like by applying thermal energy, which has been generated at the heat-generating portion, to the biological tissue as disclosed, for example, in the Japanese Patent Application JP 2002-136525 A or Patent Literature (PTL 1).

The treatment system described in PTL 1 adopts a configuration to resolve a problem of unevenly distributed load.

The term “unevenly distributed pressure load” as used herein means a state in which a biological tissue is not grasped between the entireties of grasping surfaces in a grasping portion but grasped between parts of the grasping surfaces.

For example, if a single heat-generating portion is arranged over the entirety of one of the grasping surfaces and an unevenly distributed pressure load is applied, the heat-generating portion has a temperature lower than a target temperature at a part thereof where the heat-generating portion is covered by a biological tissue because heat is transferred to the biological tissue. At another part of the heat-generating portion where the heat-generating portion is not covered by the biological tissue, on the other hand, heat is not transferred to the biological tissue, so that the other part has a temperature higher than the target temperature. Therefore, a problem arises in that the biological tissue cannot be heated at the target temperature and a longer treatment time is required.

Accordingly, the treatment system described in PTL 1 adopts a configuration that heating portions are arranged on at least one of grasping portions at respective different positions, in a longitudinal direction of the one grasping portion and the heating portions are controlled independently. Even if an unevenly distributed load is applied, a biological tissue, owing to such a configuration, can be heated at a target temperature and can be appropriately treated. With the treatment system described in PTL 1, however, a plurality of power sources is needed to supply energizing electrical power to a plurality of heating portions, respectively.

### BRIEF SUMMARY OF EMBODIMENTS

The disclosed technology has been made in view of the foregoing.

One aspect of the disclosed technology is directed to a treatment system that includes a heat generating structure element having opposed respective distal and proximal ends. The heat generating structure element includes a heat transfer member and a plurality of heat-generating portions coupled to one another. The heat transfer member is configured to transmit thermal energy to a treatment target. The plurality of heat-generating portions is coupled to the heat transfer member along a longitudinal direction extending from the distal end to the proximal end along the heat transfer member so as to transmit heat to the heat transfer member. A power source portion supplies electrical power to the plurality of heat-generating portions. A switch portion selects one target heat-generating portion from the plurality of heat-generating portions to be used as a target to which electrical power is to be supplied from the power source portion. A switch control portion is used to control operation of the switch control portion such that the one target heat-generating portion is sequentially switched from of the plurality of heat-generating portions. An energization control portion is used to control at least one of a switching timing of the target heat-generating portion by the switch control portion and the electrical power to be supplied from the power source portion to the target heat-generating portion.

Another aspect of the disclosed technology is directed to a treatment system that includes a control device and a treatment instrument configured to be attached to the control device. The treatment instrument includes a handle, a shaft, and a grasping portion for grasping and applying treatment to a treatment target. The grasping portion includes respective first and second grasping members being attached to one another. The first and second grasping members are pivotally supported on one end of the shaft so as to be opened or closed with respect to one another. The first grasping member includes a heat generating structure element having opposed respective distal and proximal ends. The heat generating structure element includes a heat transfer member and a plurality of heat-generating portions coupled to one another. The heat transfer member is configured to transmit thermal energy to the treatment target. The plurality of heat-generating portions is coupled to the heat transfer member along a longitudinal direction extending from the distal end to the proximal end along the heat transfer member so as to transmit heat to the heat transfer member. A power source portion supplies electrical power to the plurality of heat-generating portions. A switch portion selects one target heat-generating portion from the plurality of heat-generating portions to be used as a target to which electrical power is to be supplied from the power source portion. A switch control portion is used to control operation of the switch portion such that the one target heat-generating portion is sequentially switched from of the plurality of heat-generating portions. An energization control portion is used to control at least one of a switching timing of the target heat-generating portion by the switch control portion and the electrical power to be supplied from the power source portion to the target heat-generating portion.

A further aspect of the disclosed technology is directed to a method of operating a treatment system for treatment of a biological tissue. The method comprises transmitting thermal energy to the biological tissue by using a heat generating structure element having opposed respective distal and proximal ends. The heat generating structure element includes a heat transfer member and a plurality of heat-generating portions coupled to one another. Supplying electrical power to the plurality of heat-generating portions via a power source portion. Using a switch portion for selecting one target heat-generating portion from the plurality of heat-generating portions to be used as a target to which electrical power is to be supplied from the power source portion. Controlling operation of the switch portion via a switch control portion such that the one target heat-generating portion is sequentially switched from of the plurality of heat-generating portions, and implementing an energization control portion for controlling at least one of a switching timing of the target heat-generating portion by the switch control portion and wherein electrical power to be supplied from the power source portion to the target heat-generating portion.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view schematically illustrating a treatment system according to Embodiment 1 of the disclosed technology.

FIG. 2 is an enlarged view of a tip portion of the treatment system.

FIG. 3 is an exploded perspective view illustrating a heat-generating structure element.

FIG. 4 is a view of a heater as seen from the side of a heat transfer member.

FIG. 5 is a block diagram illustrating the treatment system.

FIG. 6 is a flow chart illustrating an energization control method.

FIGS. 7A-7B indicate[s] graphs illustrating a specific example of the energization control method illustrated in FIG. 6.

FIG. 8 is a view illustrating Modification 1 of Embodiment 1.

FIG. 9 is a flow chart illustrating Modification 2 of Embodiment 1.

FIG. 10 is a flow chart illustrating an energization control method in Embodiment 2 of the disclosed technology.

FIGS. 11A-11D indicate[s] graphs illustrating specific examples of the energization control method illustrated in FIG. 10.

FIGS. 12A-12B indicate graphs illustrating another specific example of the energization control method illustrated in FIG. 10.

FIGS. 13A-13C is a flow chart illustrating an energization control method in Embodiment 3 of the disclosed technology.

FIGS. 14A-14D indicate[s] graphs illustrating a specific example of the energization control method illustrated in FIGS. 13A-13C.

FIG. 15 is a block diagram illustrating a treatment system according to Embodiment 4 of the disclosed technology.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described. The disclosed technology has been made in the foregoing view, and an object thereof is to provide a treatment system that can appropriately treat a biological tissue even if an unevenly distributed load is applied and can also achieve a cost reduction.

Embodiments for carrying out the disclosed technology will hereinafter be described with reference to the drawings. It is, however, to be noted that the disclosed technology should not be limited by Embodiments to be described hereinafter. Further, like parts are designated by like numeral references in the description of the drawings.

### Embodiment 1

### Schematic Configuration of Treatment System

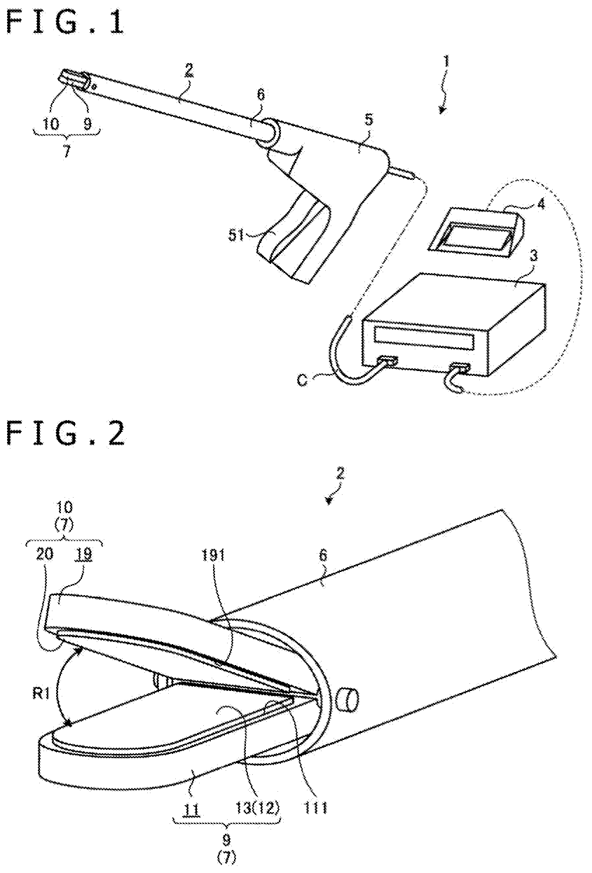

FIG. 1 is a view schematically illustrating a treatment system 1 according to Embodiment 1 of the disclosed technology.

The treatment system 1 subjects a target of treatment, which is a biological tissue, to treatment, specifically to joining or anastomosis, resection, or the like by applying thermal energy to the biological tissue. As illustrated in FIG. 1, this treatment system 1 includes a treatment instrument 2, a control device 3, and a footswitch 4.

### Configuration of Treatment Instrument

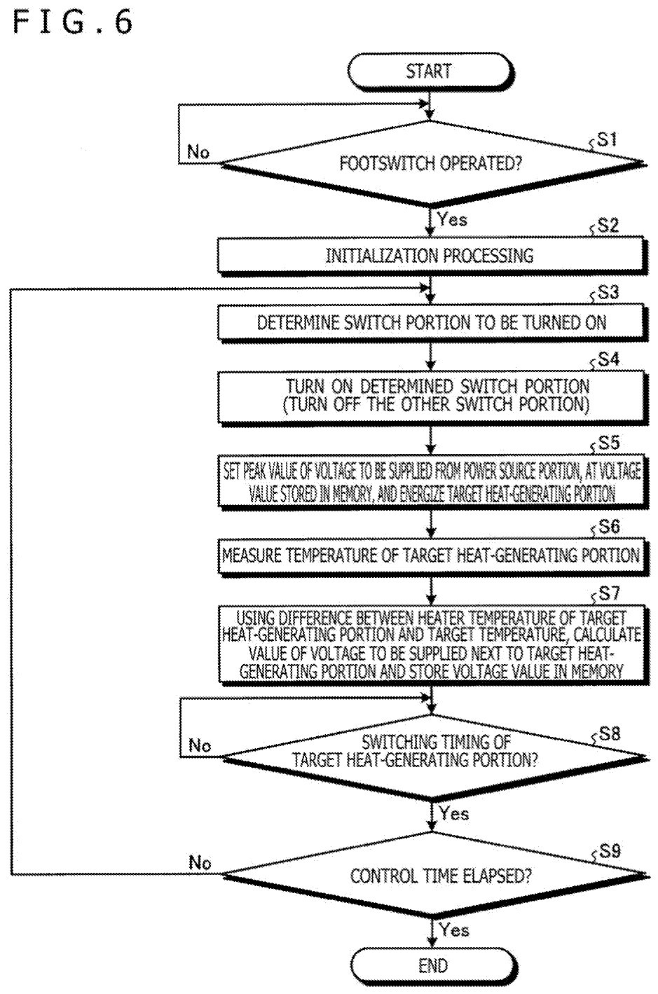

The treatment instrument 2 is, for example, a linear-type surgical instrument for applying treatment to a treatment target such as, for example, a biological tissue through the abdominal wall. As illustrated in FIG. 1, this treatment instrument 2 includes a handle 5, a shaft 6, a grasping portion 7, and a heater drive portion 8 (see FIG. 5).

The handle 5 is a portion to be held by an operator's hand. As illustrated in FIG. 1, an operation knob 51 is arranged on the handle 5.

As illustrated in FIG. 1, the shaft 6 has a tubular shape, and is connected at one of opposite ends thereof, or a first end located on its right end portion in FIG. 1, to the handle 5. Further, the grasping portion 7 is attached to a second end located on a left end portion in FIG. 1, of the shaft 6. In an interior of the shaft 6, an opening/closing mechanism (illustration omitted) is disposed to open or close a first and a second grasping member 9 and 10 (FIG. 1). The first and second grasping members 9 and 10 make up the grasping portion 7.

It is to be noted that concerning a detailed configuration of the heater drive portion 8, a description will be made upon describing configurations of the control device 3 and footswitch 4.

### Configuration of Grasping Portion

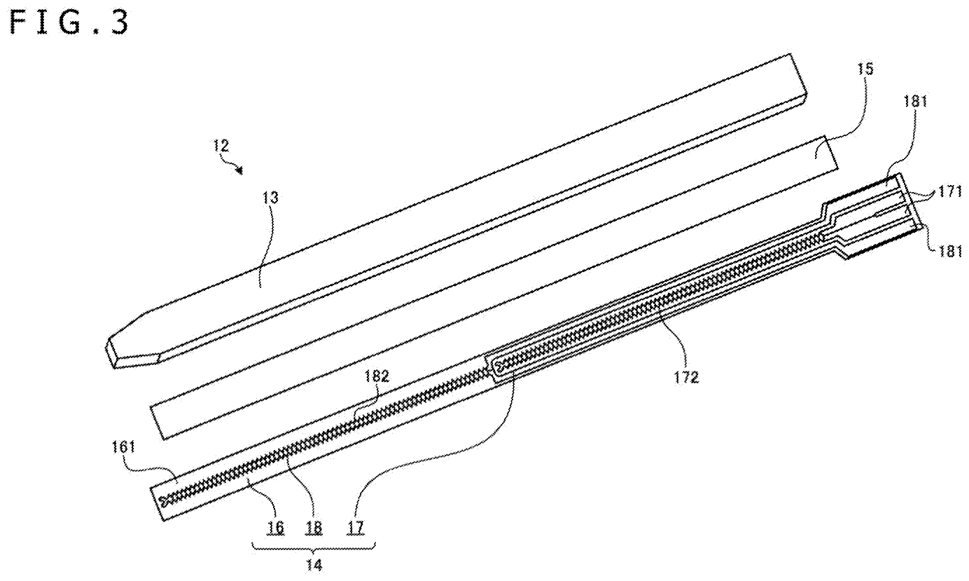

FIG. 2 is an enlarged view of a tip portion of the treatment instrument 2.

The grasping portion 7 is a portion that grasps a treatment target such as a biological tissue and treats the biological tissue. This grasping portion 7 includes the first and second grasping members 9 and 10 as illustrated in FIGS. 1 and 2.

The first and second grasping members 9 and 10 are pivotally supported on the second end of the shaft 6, or its left end portion in FIGS. 1 and 2, so that the first and second grasping members 9 and 10 can be opened or closed in a direction of an arrow R1 (FIG. 2). The first and second grasping members 9 and 10 can therefore grasp the biological tissue according to operation of the operation knob 51 by the operator.

### Configuration of First Grasping Member

It is to be noted that the term “distal end side” to be described hereinafter means a distal end side of the grasping portion 7 and a left side in FIGS. 1 and 2. It is also to be noted that the term “proximal end side” to be described hereinafter means an end of the grasping portion 7, the end being on the side of the shaft 6 and a right side in FIGS. 1 and 2.

The first grasping member 9 is disposed on a lower side relative to the second grasping member 10 in FIGS. 1 and 2. As illustrated in FIG. 2, this first grasping member 9 includes a first cover member 11 and a heat-generating structure element 12.

The first cover member 11 is configured of an elongated plate extending in a longitudinal direction from the distal end to the proximal end of the grasping portion 7 or in a left-to-right direction in FIGS. 1 and 2. In this first cover member 11, a recessed portion 111 is formed in a surface on an upper side in FIG. 2.

The recessed portion 111 is located centrally in a width direction in the first cover member 11, and extends along the longitudinal direction of the first cover member 11. Among side wall portions forming the recessed portion 111, the side wall portion on the proximal end side is omitted from illustration. The first cover member 11 supports the heat-generating structure element 12 in the recessed portion 111, and is pivotally supported on the shaft 6 in a posture that the recessed portion 111 is directed upward in FIG. 2.

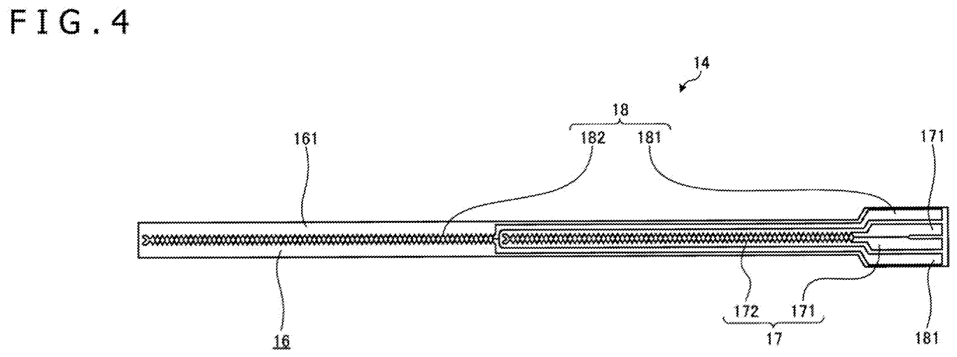

FIG. 3 is an exploded perspective view illustrating the heat-generating structure element 12. Specifically, FIG. 3 is an exploded perspective view of the heat-generating structure element 12 as viewed from an upper side in FIGS. 1 and 2.

The heat-generating structure element 12 is accommodated in the recessed portion 111 with a part thereof protruding from the recessed portion 111 to the upper side in FIG. 2. The heat-generating structure element 12 generates thermal energy under control by the control device 3. As illustrated in FIG. 3, this heat-generating structure element 12 includes a heat transfer member 13, a heater 14, and a bonding member 15.

The heat transfer member 13 is configured of a plate which is made, for example, of a material such as copper and is in an elongated form, more specifically in an elongated form extending in a longitudinal direction of the grasping portion 7.

With the biological tissue grasped by the first and second grasping members 9 and 10, the heat transfer member 13 comes at an upper-side surface thereof, as viewed in FIGS. 2 and 3, into contact with the biological tissue, and transfers heat from the heater 14 to the biological tissue, in other words, applies thermal energy to the biological tissue.

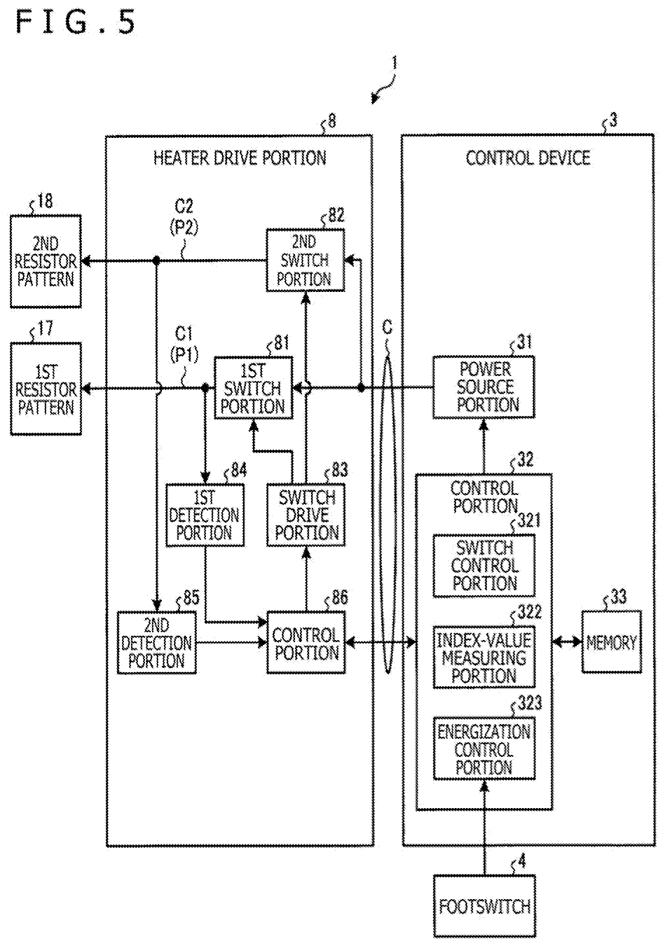

FIG. 4 is a view of the heater 14 as viewed from the side of the heat transfer member 13.

The heater 14 functions as a sheet heater, which generates heat at a part thereof and heats the heat transfer member 13 with the heat thus generated. As illustrated in FIGS. 3 and 4, this heater 14 includes a base plate 16, a first resistor pattern 17, and a second resistor pattern 18.

The base plate 16 is a sheet which is made from an insulating material such as polyimide and is in an elongated form, more specifically in an elongated form extending in the longitudinal direction of the grasping portion 7.

It is to be noted that the material of the base plate 16 is not limited to polyimide but a high heat resistant insulating material such as, for example, aluminum nitride, alumina, glass or zirconia may also be adopted without problem.

The first resistor pattern 17 has been provided by machining stainless steel, for example, SUS304 as a conductive material, and as illustrated in FIGS. 3 and 4, includes a pair of first connecting portions 171 and a first main pattern part 172. The first resistor pattern 17 has been bonded to an upper-side surface 161, as viewed in FIG. 3, of the base plate 16 by thermal press bonding.

It is to be noted that the material of the first resistor pattern 17 is not limited to stainless steel, for example, SUS304 but another stainless steel material, for example, one of 400 series or a conductive material such as platinum or tungsten may also be adopted without problem. Further, the first resistor pattern 17 is not limited to the configuration that the first resistor pattern 17 has been bonded to the surface 161 of the base plate 16 by thermal press bonding, but may also be formed on the surface 161 without problem by vapor deposition, printing or the like.

As illustrated in FIGS. 3 and 4, the paired first connecting portions 171 are each disposed on a proximal end side of the base plate 16, in other words, on the side of its right end portion in FIGS. 3 and 4, and are each arranged such that they each extend from the proximal end side toward a distal end side of the base plate 16, in other words, toward the side of its left end portion in FIGS. 3 and 4 and they face each other along a width direction of the base plate 16. To the paired first connecting portions 171, two first leads C1 (see FIG. 5) are joined or connected, respectively. The first leads C1 are connected to the heater drive portion 8, and are laid from the side of the one end of the shaft 6, or the side of its right end portion in FIG. 1, to the side of the other end thereof, or the side of its left end portion in FIG. 1, in an interior of the shaft 6. It is to be noted that, in FIG. 5, only one of the first leads C1 is illustrated for the sake of convenience of the description.

The first main pattern part 172 is connected or in conduction at an end thereof to one of the first connecting portions 171, extends from the end thereof toward the distal end side of the base plate 16 while meandering in a waveform pattern, is folded back around an approximately longitudinal center of the base plate 16 toward the proximal end side of the base plate 16, and is connected or in conduction at its opposite end to the other first connecting portion 171. Also, the first main pattern part 172 has a resistance value set greater than that of the paired first connecting portions 171, both per unit length in the longitudinal direction of the base plate 16.

The first main pattern part 172 generates heat by a voltage impressed or applied across the paired first connecting portions 171 via the two first leads C1 by the heater drive portion 8. Therefore, the first main pattern part 172 corresponds to the heat-generating portion in the disclosed technology.

The second resistor pattern 18 has been provided by machining stainless steel, for example, SUS304 as a conductive material, and as illustrated in FIGS. 3 and 4, includes a pair of second connecting portions 181 and a second main pattern part 182. The second resistor pattern 18 has been bonded to the surface 161 of the base plate 16 by thermal press bonding.

It is to be noted that the material of the second resistor pattern 18 is not limited to stainless steel, for example, SUS304 but another stainless steel material, for example, one of 400 series or a conductive material such as platinum or tungsten may also be adopted without problem. Further, the second resistor pattern 18 is not limited to the configuration that the second resistor pattern 18 has been bonded to the surface 161 of the base plate 16 by thermal press bonding, but may also be formed on the surface 161 without problem by vapor deposition, printing or the like. Also, the material of the second resistor pattern 18 can be the same as the material of the first resistor pattern 17, or can be changed to a different material without problem.

As illustrated in FIGS. 3 and 4, the paired second connecting portions 181 are arranged such that they each extend from the proximal end side of the base plate 16 to around an approximately longitudinal center of the base plate 16 and they face each other along the width direction of the base plate 16 with the first resistor pattern 17 interposed therebetween. To the paired second connecting portions 181, two second leads C2 (see FIG. 5) are joined or connected, respectively. The second leads C2 are connected to the heater drive portion 8, and are laid from the side of the one end of the shaft 6, or the side of its right end portion as viewed in FIG. 1, to the side of the other end thereof, or the side of its left end portion as viewed in FIG. 1, in the interior of the shaft 6. It is to be noted that, in FIG. 5, only one of the second leads C2 is illustrated for the sake of convenience of the description.

The second main pattern part 182 is connected or in conduction at one of opposite ends thereof to one of the second connecting portions 181, extends from the one end thereof to the distal end of the base plate 16 while meandering in a waveform pattern, is folded back at the distal end toward the proximal end of the base plate 16, and is connected or in conduction to the other second connecting portion 181. Also, the second main pattern part 182 has a resistance value set greater than that of the paired second connecting portions 181, both per unit length in the longitudinal direction of the base plate 16.

The second main pattern part 182 generates heat by a voltage impressed or applied across the paired second connecting portions 181 via the two second leads C2 by the heater drive portion 8. Therefore, the second main pattern part 182 also corresponds to the heat-generating portion in the disclosed technology.

As described hereinbefore, the first and second main pattern parts 172 and 182 are disposed side by side in the longitudinal direction of the base plate 16, in other words, are disposed at different respective positions, in the longitudinal direction.

As illustrated in FIG. 3, the bonding member 15 is interposed between the heat transfer member 13 and the surface 161 of the base plate 16 or the first and second resistor patterns 17 and 18, and fixedly bonds the heat transfer member 13 and the heater 14. This bonding member 15 is configured of a sheet, which is in an elongated form, specifically in an elongated form extending in the longitudinal direction of the grasping portion 7, has good thermal conductivity and electrical insulating property, is resistant to high temperatures, and has bonding property.

As also illustrated in FIG. 3, the heat transfer member 13 is disposed so as to cover the entirety of the first and second main pattern parts 172 and 182. Meanwhile, the bonding member 15 is disposed so as to cover the entirety of the first and second main pattern parts 172 and 182 and cover parts of the respective, paired first connecting portions 171 and paired second connecting portions 181. In other words, the bonding member 15 is disposed in a state that it extends toward the proximal end relative to the heat transfer member 13. The two first leads C1 and two second leads C2 are connected or joined to regions of the paired first connecting portions 171 and paired second connecting portions 181, respectively, the regions being not covered by the bonding member 15.

### Configuration of Second Grasping Member

As illustrated in FIG. 2, the second grasping member 10 includes a second cover member 19 and an opposing plate 20.

The second cover member 19 has the same shape as the first cover member 11. Specifically, the second cover member 19 has a recessed portion 191 similar to the recessed portion 111 as illustrated in FIG. 2. The second cover member 19 supports the opposing plate 20 in the recessed portion 191, and is pivotally supported on the shaft 6 in a posture that the recessed portion 191 is directed downward in FIG. 2 or in a posture that the recessed portion 191 opposes the recessed portion 111.

The opposing plate 20 is configured, for example, of a conductive material such as copper. This opposing plate 20 is configured of a flat plate having substantially the same planar shape as the recessed portion 191, and is fixedly secured in the recessed portion 191. The opposing plate 20 grasps a biological tissue between itself and the heat transfer member 13.

It is to be noted that without being limited to such a conductive material, the opposing plate 20 may also be configured of another material, for example, a resin material such as polyether ether ketone (PEEK) without problem.

### Configurations of Control Device and Footswitch

FIG. 5 is a block diagram illustrating the treatment system 1.

The footswitch 4 is an element to be operated by the operator's foot. According to the operation to the footswitch 4, the control device 3 performs energization control of the heater 14 or the first and second resistor patterns 17 and 18.

It is to be noted that means for causing to perform the energization control is not limited to the footswitch 4, but a hand-operated switch or the like may also be adopted instead without problem.

The control device 3 is composed including a central processing unit (CPU) or the like, and comprehensively controls operation of the treatment instrument 2 according to a predetermined control program. As illustrated in FIG. 5, this control device 3 includes a power source portion 31, a control portion 32, and a memory 33.

The power source portion 31 is connected to the heater drive portion 8 via an electrical cable C (see FIGS. 1 and 5). For the energization of the first and second resistor patterns 17 and 18, the power source portion 31 supplies electrical power to the heater drive portion 8 via the electrical cable C under control by the control portion 32.

The control portion 32 is configured, for example, of a CPU or the like. The control portion 32 controls operation of the power source portion 31. The control portion 32 also performs communication with the heater drive portion 8 via the electrical cable C to control operation of the heater drive portion 8. As illustrated in FIG. 5, this control portion 32 includes a switch control portion 321, an index-value measuring portion 322, and an energization control portion 323.

It is to be noted that, concerning functions of the switch control portion 321, index-value measuring portion 322 and energization control portion 323, a description will be made after making a description about the configuration of the heater drive portion 8.

The memory 33 stores the control program to be executed by the control portion 32, data required in processing by the control portion 32, and the like. Here, illustrative examples of the data required in the processing by the control portion 32 include resistance-temperature characteristic information indicating a relation between resistance values and temperatures at each of the first and second resistor patterns 17 and 18, energizing voltage values to the first and second resistor patterns 17 and 18, and the like.

The heater drive portion 8 is arranged, for example, in the interior of the handle 5. As illustrated in FIG. 5, the heater drive portion 8 includes a first and second switch portions 81 and 82, a switch drive portion 83, a first and second detection portions 84 and 85, and a control portion 86.

The first switch portion 81 is configured, for example, of a field effect transistor (FET) or the like, and is arranged in a supply route of electrical power to the first resistor pattern 17 (hereinafter described as a “first supply route P1” (see FIG. 5)). The first supply route P1 connects the electrical cable C and the first resistor pattern 17 or the first lead C1. If turned on by the switch drive portion 83, the first switch portion 81 allows to supply electrical power to the first resistor pattern 17 or allows energization of the first resistor pattern 17 via the first supply route P1. If turned off, conversely, the first switch portion 81 prohibits the supply of electrical power to the first resistor pattern 17 or prohibits energization of the first resistor pattern 17 via the first supply route P1.

The second switch portion 82 is configured, for example, of an FET or the like, and is arranged in a supply route of electrical power to the second resistor pattern 18 (hereinafter described as a “second supply route P2 (see FIG. 5)). The second supply route P2 connects the electrical cable C and the second resistor pattern 18 or the second lead C2. If turned on by the switch drive portion 83, the second switch portion 82 allows to supply electrical power to the second resistor pattern 18 or allows energization of the second resistor pattern 18 via the second supply route P2. If turned off, conversely, the second switch portion 82 prohibits the supply of electrical power to the second resistor pattern 18 or prohibits energization of the second resistor pattern 18 via the second supply route P2.

If the first switch portion 81 is turned on and the second switch portion 82 is turned off, the first resistor pattern 17 is selected as a single target heat-generating portion which is a target to be supplied with electrical power from the power source portion 31. Conversely, if the first switch portion 81 is turned off and the second switch portion 82 is turned on, the second resistor pattern 18 is selected as a single target heat-generating portion which is a target to be supplied with electrical power from the power source portion 31. Therefore, the first and second switch portions 81 and 82 select one of the first and second resistor patterns 17 and 18 as a single target heat-generating portion, and correspond to the switch portion in the disclosed technology.

The switch drive portion 83 turns on or turns off the first and second switch portions 81 and 82 under control by the control portion 86.

The first detection portion 84 is connected to the first supply route P1, and detects the values of current and voltage to be supplied to the first resistor pattern 17. The first detection portion 84 then outputs, to the control portion 86, detection signals corresponding to the current value and voltage value so detected.

The second detection portion 85 is connected to the second supply route P2, and detects the values of current and voltage to be supplied to the second resistor pattern 18. The second detection portion 85 then outputs, to the control portion 86, detection signals corresponding to the current value and voltage value so detected.

The control portion 86 is configured, for example, of a CPU or the like, and performs communication with the control portion 32 of the control device 3 via the electrical cable C. The control portion 86 transmits the detection signals, which have been detected by the first and second detection portions 84 and 85, to the control portion 32 via the electrical cable C, and controls operation of the switch drive portion 83 according to control signals transmitted from the control portion 32.

The switch control portion 321 transmits the control signals to the control portion 86 via the electrical cable C to control operation of the first and second switch portions 81 and 82, whereby the single target heat-generating portion is sequentially switched between the first and second resistor patterns 17 and 18.

Based on the detection signals or the values of current and voltage, which are to be supplied to the first and second resistor patterns 17 and 18, as transmitted from the control portion 86 via the electrical cable C, the index-value measuring portion 322 calculates resistance values of the first and second resistor patterns 17 and 18. Based on the resistance-temperature characteristic information corresponding to the first and second resistor patterns 17 and 18, respectively, and stored in the memory 33, the index-value measuring portion 322 converts the calculated resistance values to temperatures of the first and second resistor patterns 17 and 18, respectively.

Based on the temperatures of the first and second resistor patterns 17 and 18 as measured by the index-value measuring portion 322, the energization control portion 323 controls at least one of switching timing of the target heat-generating portion by the switch control portion 321 and electrical power to be supplied from the power source portion 31 to the target heat-generating portion.

### Energization Control Method

A description will next be made about operation or an energization control method of the treatment system 1 described hereinbefore.

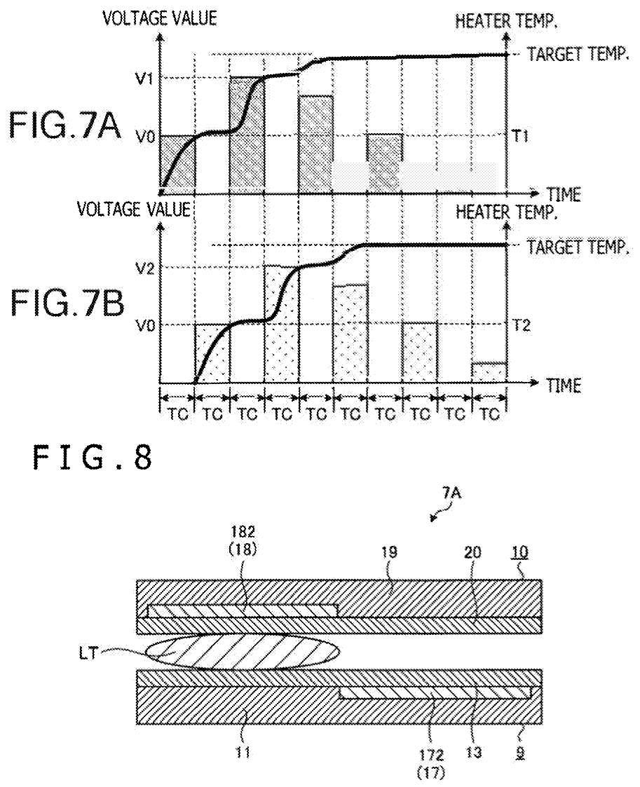

FIG. 6 is a flow chart illustrating the energization control method.

The operator holds the treatment instrument 2 with his or her hand, and inserts a tip portion of the treatment instrument 2 or the grasping portion 7 and a part of the shaft 6 into the abdominal cavity through the abdominal wall by using, for example, a trocar or the like. The operator then operates the operation knob 51 to grasp a biological tissue as a target of treatment by the grasping portion 7.

According to operation of the footswitch 4 by the operator in Yes in Step S1, the control device 3 then performs energization control as will be described hereinafter.

First, the control portion 32 performs initialization processing in Step S2. In Step S2, the control portion 32 stores, for example, the values of initial voltages, which are to be applied across the first and second resistor patterns 17 and 18, as the values of energizing voltages to the first and second resistor patterns 17 and 18 in the memory 33.

After Step S2, the switch control portion 321 determines, out of the first and second switch portions 81 and 82, the switch portion to be turned on in Step S3. In a case where the first switch portion 81 has been determined, in an immediately preceding loop or a loop of Steps S3 to S9, as the switch portion to be turned on, for example, the second switch portion 82 is determined, in the next loop, as the switch portion to be turned on.

After Step S3, the switch control portion 321 turns on the switch portion, which has been determined in Step S3, out of the first and second switch portions 81 and 82, and turns off the other switch portion in Step S4. Therefore, out of the first and second resistor patterns 17 and 18, the resistor pattern connected to the turned-on switch portion is selected as a target heat-generating portion.

After Step S4, the energization control portion 323 reads, from the memory 33, the energizing voltage value corresponding to the target heat-generating portion selected in Step 4, or the initial voltage value stored in the memory 33 in Step S2 or a voltage value stored in the memory 33 in Step S7. The energization control portion 323 then controls operation of the power source portion 31, sets the peak value of a voltage, which is to be supplied from the power source portion 31, at the voltage value so read, and energizes the target heat-generating portion at the voltage value in step S5. It is to be noted that, in the first loop or the loop of Steps S3 to S9, the energization control portion 323 reads the initial voltage value stored in the memory 33 in Step S2 and energizes the target heat-generating portion at the initial voltage value.

After Step S5, the index-value measuring portion 322 measures, in step S6, the temperature of the target heat-generating portion (hereinafter described as “the heater temperature”) based on a detection signal from one of the first and second detection portions 84 and 85, the one detection portion being connected to the target heat-generating portion selected in Step S4.

After Step S6, the energization control portion 323 calculates the value of a voltage, which is to be next applied to the target heat-generating portion, by using the difference between the heater temperature of the target heat-generating portion as measured in Step S6 and a target temperature, and in the memory 33, stores the calculated voltage value or updates to the calculated voltage value as the value of an energizing voltage to the target heat-generating portion in Step S7. It is to be noted that, upon calculation of the voltage value, commonly-used proportional-integral-differential (PID) control or the like is used.

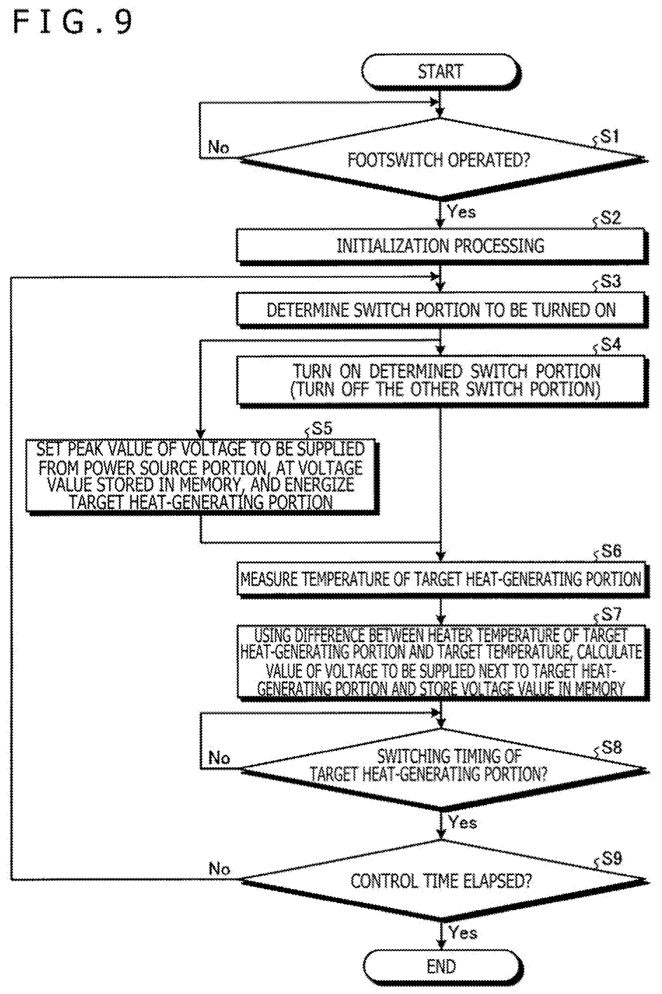

After Step S7, the energization control portion 323 continually monitors in step S8 whether or not the switching timing of the target heat-generating portion has been reached. Specifically, the energization control portion 323 determines a time point at which a predetermined time TC (see FIGS. 7A-7B) has elapsed since the starting of energization of the target heat-generating portion in Step S5, as a switching timing in Step S8. Therefore, the switching timing is set with a constant interval in Embodiment 1.

In Embodiment 1, the predetermined time TC is set to be equal to or shorter than the time constant of a temperature change of the target heat-generating portion. Here, the term “time constant” is time until the change occurs in the heater temperature, and means, for example, a time from the beginning of the heater temperature to lower from a state that the energization of the target heat-generating portion has ended until it is lowering to a predetermined value. In a case where the predetermined time TC is set at a time longer than the time constant, a biological tissue cannot be treated or heated appropriately or a deterioration may occur in treatment performance or speed, so that control to the target temperature is needed. The time constant significantly varies depending on the target tissue such as the stomach, the blood vessel, the intestine or the specification, for example, the construction, material and the like of the device. In other words, the term “time constant” more specifically means the time until the target heat-generating portion lowers to 291° C. in a case where the target heat-generating portion is controlled at 300° C. with the predetermined value being set, for example, within +3%. In this embodiment, the predetermined time TC is set at 20 ms.

In a case where the switching timing of the target heat-generating portion is determined to have been reached in Yes in Step S8, the control portion 32 determines in Step S9 whether or not the treatment time required for the treatment of the biological tissue has elapsed. Specifically, the control portion 32 determines in Step S9 whether or not the predetermined time has elapsed since the operation of the footswitch 4 in Yes in Step S1.

If the treatment time is determined to have elapsed in Yes in Step S9, the control device 3 ends the energization control.

If the treatment time is determined not to have elapsed, conversely, in No in Step S9, the control device 3 returns the processing to Step S3.

### Specific Example of Energization Control Method

A description will next be made about a specific example of the energization control method described hereinbefore.

FIGS. 7A-7B indicate graphs illustrating the specific example of the energization control method. Specifically, FIG. 7A is a graph illustrating changes in the heater temperature and the voltage value during energization at the first resistor pattern 17. FIG. 7B is a graph illustrating changes in the heater temperature and the voltage value during energization at the second resistor pattern 18. It is to be noted that FIGS. 7A-7B exemplify a case in which the first switch portion 81 is turned on first. In FIGS. 7A-7B, heater temperatures are expressed by a line graph while voltage values are expressed by a bar graph.

In a first loop of Steps S3 to S9, the first resistor pattern 17 is selected as a target heat-generating portion in Step S4. As illustrated in FIG. 7A, the first resistor pattern 17 is then energized at an initial voltage value V0 in Step S5. During the energization, for example, at a timing immediately before ending the energization, the first resistor pattern 17 is measured for a heater temperature T1 in Step S6, and using the heater temperature T1, the value V1 of a voltage to be next supplied to the first resistor pattern 17 or to be supplied in a third loop of Steps S3 to S9 is calculated in Step S7. If the predetermined time TC has elapsed since the starting of the energization of the first resistor pattern 17 in Yes in Step S8, the target heat-generating portion is switched from the first resistor pattern 17 to the second resistor pattern 18 in Step S3. As a consequence, the first loop of Steps S3 to S9 is ended.

In a second loop of Steps S3 to S9, the second resistor pattern 18 is selected as a target heat-generating portion in Step S4. As illustrated in FIG. 7B, the second resistor pattern 18 is then energized at the initial voltage value V0 in Step S5. During the energization, for example, at a timing immediately before ending the energization, the second resistor pattern 18 is measured for a heater temperature T2 in Step S6, and using the heater temperature T2, the value V2 of a voltage to be next supplied to the second resistor pattern 18 or to be supplied in a fourth loop of Steps S3 to S9 is calculated in Step S7. If the predetermined time TC has elapsed since the starting of the energization of the second resistor pattern 18 in Yes in Step S8, the target heat-generating portion is switched from the second resistor pattern 18 to the first resistor pattern 17 in Step S3. As a consequence, the second loop of Steps S3 to S9 is ended.

By repeatedly performing the loop of S3 to S9, the heater temperatures of the first and second resistor patterns 17 and 18 are therefore each controlled to the target temperature as illustrated in FIGS. 7A-7B.

According to the Embodiment 1 described hereinbefore, the following advantageous effects are brought about.

In the treatment system 1 according to Embodiment 1, the first and second main pattern parts 172 and 182 are arranged at the different respective positions, in the longitudinal direction of the grasping portion 7, and are controlled independently.

Even if an unevenly distributed load is applied as in the configuration described in PTL 1, it is therefore possible to heat a biological tissue at a target temperature and to appropriately treat the biological tissue.

Also, in the treatment system 1 according to Embodiment 1, the first and second resistor patterns 17 and 18 are independently controlled owing to the switching of the supply routes of electrical power or the first and second supply routes P1 and P2 from the power source portion 31 to the first and second resistor patterns 17 and 18 or the first and second main pattern parts 172 and 182 by the first and second switch portions 81 and 82.

Compared with the configuration described in PTL 1, it is therefore unnecessary to arrange a plurality of power source portions 31 and possible to achieve a cost reduction.

As described hereinbefore, the treatment system 1 according to Embodiment 1 brings about advantageous effects that a biological tissue can be appropriately treated even under an unevenly distributed load and a cost reduction can be achieved.

In the treatment system 1 according to Embodiment 1, the time from stopping a supply of electrical power until starting a next supply of electrical power with respect to the target heat-generating portion, that is, the predetermined time TC is set to become not greater than the time constant of temperature changes at the target heat-generating portion.

It is therefore possible to make substantially equal the heater temperature of a target heat-generating portion at the time point of stopping the supply of electrical power to the target heat-generating portion and the heater temperature of the next target heat-generating portion at the time point of staring the next supply of electrical power (see, for example, heater temperatures T1 and T2 indicated in FIGS. 7A-7B). In other words, a voltage value upon supplying electrical power next, for example, the voltage value V1 or V2 indicated in FIGS. 7A-7B can be appropriately calculated using the heater temperature, for example, the heater temperature T1 or T2 indicated in FIGS. 7A-7B of the target heat-generating portion at the time point of stopping the supply of electrical power to the target heat-generating portion. Accordingly, the heater temperatures of the first and second resistor patterns 17 and 18 can be controlled appropriately to and stably at the target temperature.

### Modification 1 of Embodiment 1

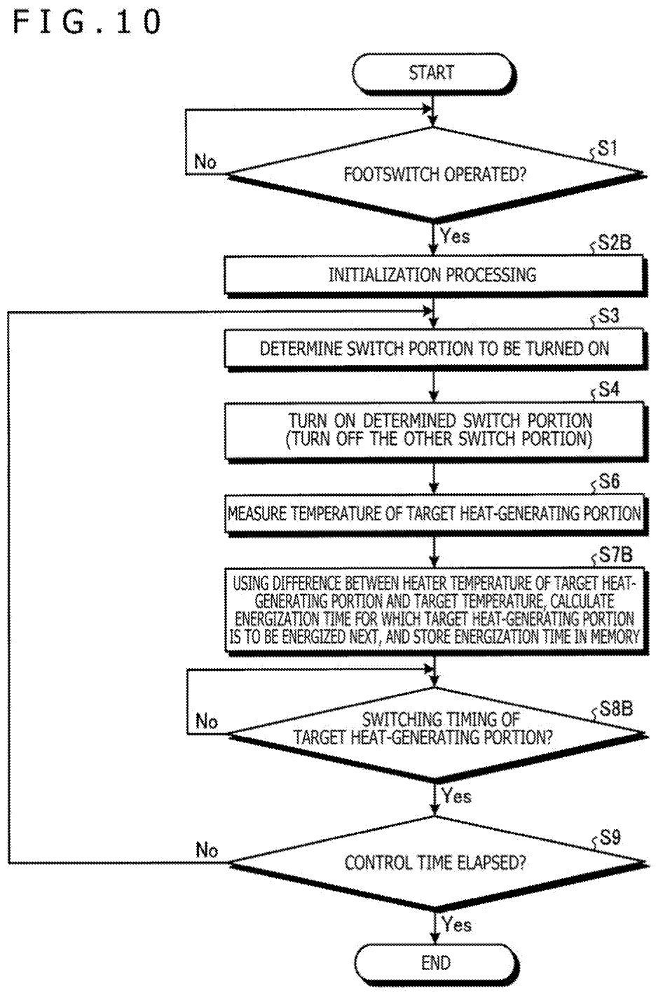

FIG. 8 is a view illustrating Modification 1 of Embodiment 1. Specifically, FIG. 8 is a cross-sectional view of a grasping portion 7A in Modification 1, taken along a plane intersecting at right angles to a width direction of the grasping portion 7A in a state that the grasping portion 7A is closed or in a state that a biological tissue LT is grasped by the grasping portion 7A. It is to be noted that, in FIG. 8, the paired first connecting portions 171 and the paired second connecting portions 181 are omitted from the illustration for the sake of convenience of the description.

In Embodiment 1 described hereinbefore, the first and second main pattern parts 172 and 182 are disposed side by side in the longitudinal direction on the first grasping member 9. However, the first and second main pattern parts 172 and 182 may also be disposed without problem as illustrated in FIG. 8 insofar as they are arranged at positions different in the longitudinal direction.

Specifically, in the grasping portion 7A in Modification 1, the first resistor pattern 17 is disposed on the first grasping member 9 as illustrated in FIG. 8. Meanwhile, the second resistor pattern 18 is disposed on the second grasping member 10. In addition, the first and second main pattern parts 172 and 182 are arranged at respective positions different in the longitudinal direction.

The adoption of the configuration of Modification 1 described hereinbefore brings about similar advantageous effects as in Embodiment 1 described hereinbefore.

### Modification 2 of Embodiment 1

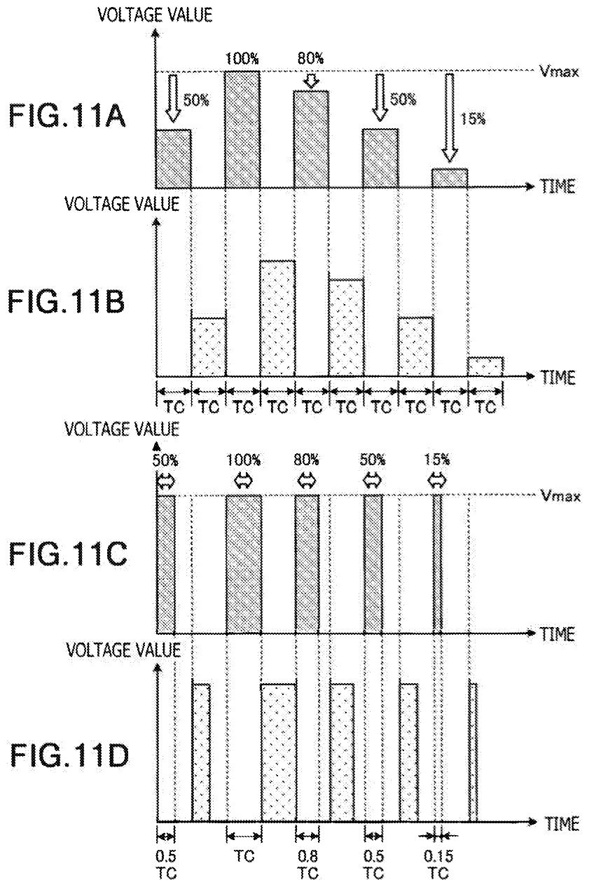

FIG. 9 is a flow chart illustrating Modification 2 of Embodiment 1.

In Embodiment 1 described hereinbefore, Step S4 and Step S5 may also be performed at the same time, in other words, subjected to parallel processing as illustrated in FIG. 9.

According to Modification 2 described hereinbefore, no time difference takes place between the switching between the first and second switch portions 81 and 82 in Step S4 and the energization of the target heat-generating portion in Step S5, so that energization control can be performed with higher accuracy.

### Embodiment 2

A description will next be made about Embodiment 2.

In the following description, similar configurations and steps as in Embodiment 1 described hereinbefore will be identified by the same numeral references, and their detailed description will be omitted or simplified.

In Embodiment 1 described hereinbefore, the peak value of a voltage to be supplied from the power source portion 31 is controlled while setting the switching timing with the constant interval.

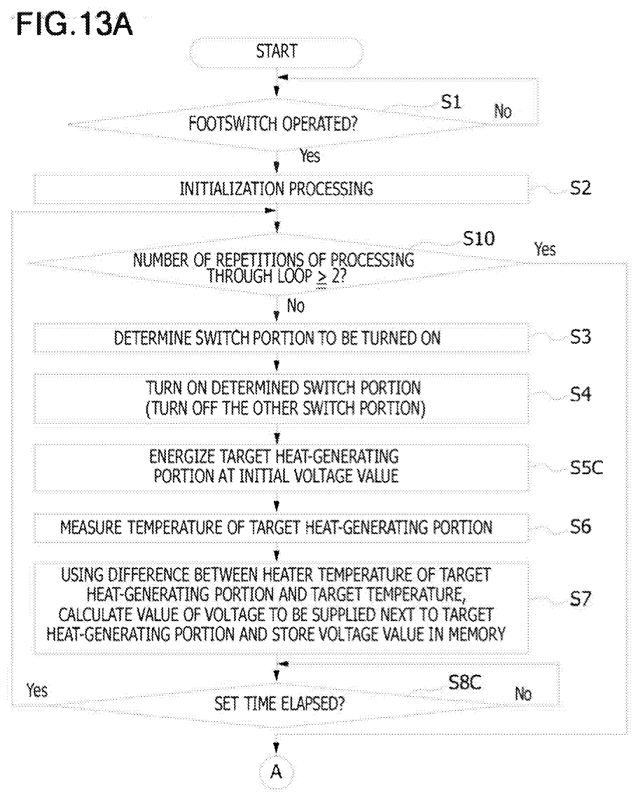

In Embodiment 2, in contrast, the energization time for which the target heat-generating portion is continually energized is controlled while maintaining constant the peak value of a voltage, which is to be supplied from the power source portion 31, specifically at a predetermined voltage value Vmax (see FIGS. 11C and 11D). From Embodiment 1 described hereinbefore, Embodiment 2 is therefore different in the energization control method.

### Energization Control Method

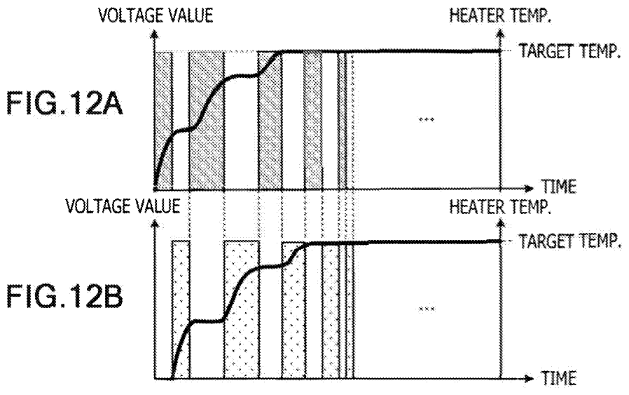

FIG. 10 is a flow chart illustrating the energization control method in Embodiment 2.

As illustrated in FIG. 10, the energization control method in Embodiment 2 is different from the energization control method of FIG. 6 as described in Embodiment 1 described hereinbefore in that Step S5 is omitted and Steps S2B, S7B, and S8B are adopted instead of Steps S2, S7, and S8. It is to be noted that, as Step S5 has been omitted in Embodiment 2, Step S6 is performed after Step S4. Only Steps S2B, S7B, and S8B will be described hereinafter.

If the footswitch 4 has been operated by the operator in Yes in Step S1, Step S2B is performed.

Specifically, the energization control portion 323 causes the power source portion 31 to operate and to supply a voltage of the predetermined voltage value Vmax from the power source portion 31 in Step S2B. The control device 3 then allows the energization control processing to proceed to Step S3.

By the performance of Step S2B, the target heat-generating portion selected in Step S4 is energized at the predetermined voltage value Vmax.

Step S7B is performed after Step S6.

In Step S7B, the energization control portion 323, similar to Step S7 described in Embodiment 1 described hereinbefore, calculates the value of a voltage, which is to be next supplied to the target heat-generating portion, by using the difference between the heater temperature of the target heat-generating portion as measured in Step S6 and the target temperature. The energization control portion 323 also calculates the percentage of the calculated voltage value to the predetermined voltage value Vmax. As an energization time for which the target heat-generating portion is to be energized next, the energization control portion 323 then calculates time corresponding to the calculated percentage of the predetermined time TC, and stores the calculated energization time in the memory 33.

After Step S7B, the energization control portion 323 continually monitors in Step S8B whether or not the switching timing of the target heat-generating portion has been reached. Specifically, in Step S8B, the energization control portion 323 reads the energization time stored in the memory 33 in the twice-preceding loop, that is, in the loop of Steps S3, S4, S6, S7B, S8B, and S9, and sets, as a switching timing, a time point at which the energization time has elapsed since the starting of the energization of the target heat-generating portion in Step S4. In a case where it is determined that the switching timing has been reached or the energization time has elapsed in Yes in Step S8B, the control device 3 allows the energization control processing to proceed to Step S9.

Specific Example of Energization Control Method A description will next be made about a specific example of the energization control method in Embodiment 2.

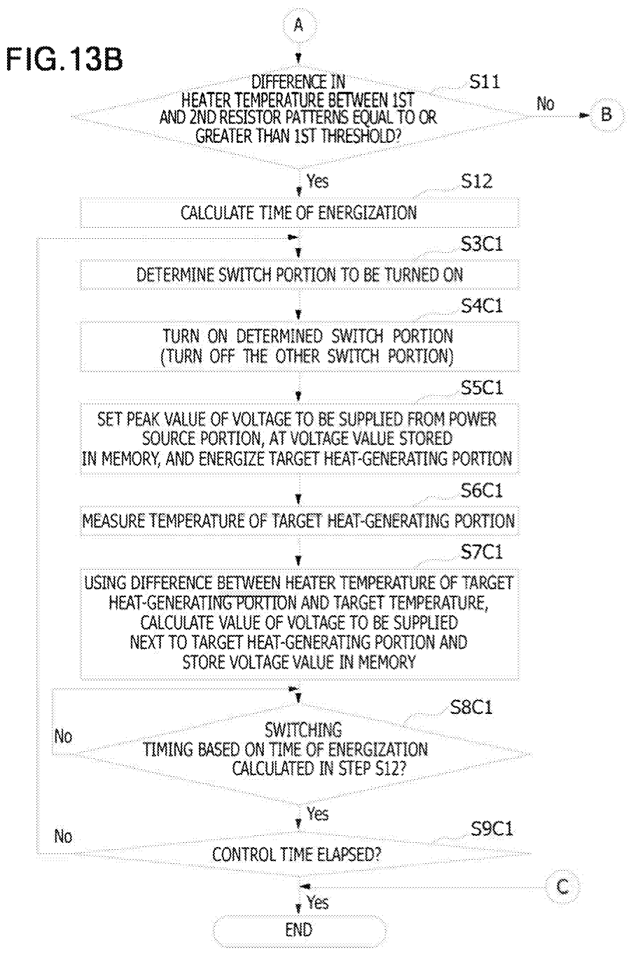

FIGS. 11A-11D and 12A-12B are graphs illustrating the specific examples of the energization control method. Specifically, FIGS. 11A and 11B illustrate changes in voltage value during energization of the first and second resistor patterns 17 and 18, respectively, when energization control is performed by the energization control method (hereinafter described as “the LEVEL method”) described hereinbefore in Embodiment 1. FIGS. 11C and 11D illustrate changes in energization time at the first and second resistor patterns 17 and 18, respectively, when energization control is performed by the energization control method (hereinafter described as “the PWM method”) described in Embodiment 2. It is to be noted that, in FIGS. 11C and 11D, the switching timing of the target heat-generating portion is set same as the switching timing in the LEVEL method in FIGS. 11A and 11B for the sake of convenience of the description. FIGS. 12A-12B corresponds to FIGS. 7A-7B. It is also to be noted that FIGS. 11A and 11C and FIG. 12A illustrate changes in voltage value and energization time during energization of the first resistor pattern 17. It is also to be noted that FIGS. 11B and 11D and FIG. 12B illustrate changes in voltage value and energization time during energization of the second resistor pattern 18.

In Embodiment 2, the value of a voltage to be supplied to the first and second resistor patterns 17 and 18 is constant at the predetermined voltage value Vmax as illustrated in FIGS. 11C and 11D. Here, the predetermined voltage value Vmax is set, for example, to be the value of a maximum voltage to be supplied to the first and second resistor patterns 17 and 18 in Embodiment 1 described hereinbefore.

A case is now assumed in which, as illustrated in FIG. 11A, voltage values in the LEVEL method as calculated in Step S7 are percentages of 50%, 100%, 80%, 50% and 15% of the predetermined voltage value Vmax.

In Step S7B, the energization time is calculated as times corresponding to the percentages of the predetermined time TC. In this assumed case, the energization time is therefore calculated, as illustrated in FIG. 11C, to be 0.5TC (if the calculated voltage value is 50% of the voltage value Vmax), TC (if the calculated voltage value is 100% of the voltage value Vmax), 0.8TC (if the calculated voltage value is 80% of the voltage value Vmax), 0.5TC (if the calculated voltage value is 50% of the voltage value Vmax), and 0.15TC (if the calculated voltage value is 15% of the voltage value Vmax), respectively.

The target heat-generating portion is then switched every energization time in Step S8B and Step 3, whereby the heater temperatures of the first and second resistor patterns 17 and 18 are each controlled to the target temperature as illustrated in FIGS. 12A-12B.

According to Embodiment 2 described hereinbefore, the following advantageous effects are brought about in addition to advantageous effects similar to those available from Embodiment 1 described hereinbefore.

In the treatment system 1 according to Embodiment 2, the energization control portion 323 maintains constant, specifically constant at the predetermined voltage value Vmax the peak value of electrical power to be supplied from the power source portion 31 to the target heat-generating portion, and based on the peak temperature of the target heat-generating portion, controls the energization time for which the target heat-generating portion is to be continually energized.

As the power source portion 31, a configuration to fix an output value can hence be adopted instead of a configuration to make an output value variable. Accordingly, it is possible to achieve a still further cost reduction of the treatment system 1.

### Embodiment 3

A description will next be made about Embodiment 3.

In the following description, similar configurations and steps as in Embodiment 1 described hereinbefore will be identified by the same numeral references, and their detailed description will be omitted or simplified.

In Embodiment 3, different from Embodiment 1 described hereinbefore, the position of the biological tissue LT in the state that the biological tissue LT is grasped by the grasping portion 7 is discriminated, and energization control of the heater 14 or the first and second resistor patterns 17 and 18 is performed according to the position. Embodiment 3 is therefore different in the energization control method from Embodiment 1 described hereinbefore.

### Energization Control Method

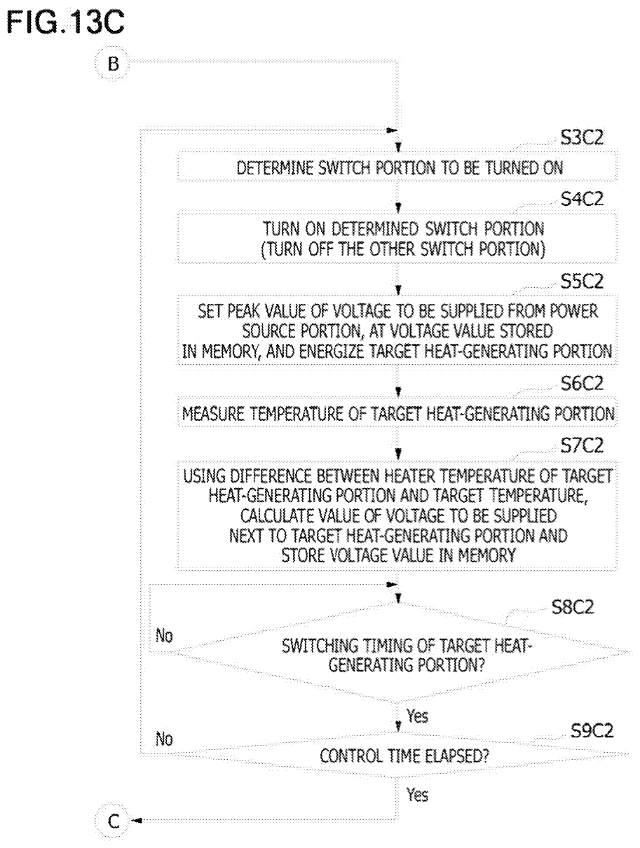

FIGS. 13A-13C is a flow chart illustrating the energization control method in Embodiment 3.

As illustrated in FIGS. 13A-13C, the energization control method in Embodiment 3 is different from the energization control method of FIG. 6 as described in Embodiment 1 described hereinbefore in that Steps S5C, S8C, S9C1, and S9C2 are adopted instead of Steps S5, S8, and S9 and Steps S10 to S12, S3C1 to S8C1, and S3C2 to S8C2 are added. Only Steps S10 to S12, S5C, S8C, S3C1 to S9C1, and S3C2 to S9C2 will be described hereinafter.

Step S10 is performed after step S2.

Specifically, the control portion 32 determines in Step S10 whether or not the processing of a loop of Steps S3, S4, S5C, S6, S7, S8C, and S10 has been performed twice.

If the processing of the loop is determined not to have been performed twice in No in Step S10, the control device 3 allows the energization control processing to proceed to Step S3.

Step S5C is performed after Step S4.

Specifically, the energization control portion 323, in Step S5C, controls operation of the power source portion 31, sets the peak value of voltage, which is to be supplied from the power source portion 31, at the initial voltage value stored in memory 33 in Step S2, and energizes the target heat-generating portion at the initial voltage value. The control device 3 then allows the energization control processing to proceed to Step S6.

Step S8C is performed after Step S7.

Specifically, taking, as a switching timing, a time point at which a set time, for example, the predetermined time TC has elapsed since the starting of the energization of the target heat-generating portion in Step S5C, the energization control portion 323 continually monitors in Step S8C whether or not the switching timing has been reached. In a case where it is determined that the switching timing has been reached in Yes in Step S8C, the control device 3 returns the energization control processing to Step S10.

Specifically, the processing of the loop of Steps S3, S4, S5C, S6, S7, S8C, and S10 is performed twice, whereby the heater temperature of the first resistor pattern 17 when the first resistor pattern 17 has been energized at the initial voltage value only for the set time, for example, the predetermined time TC and the heater temperature of the second resistor pattern 18 when the second resistor pattern 18 has been energized at the initial voltage value only for the set time, for example, the predetermined time TC are measured, respectively.

Step S11 is performed in a case where the processing of the loop of Steps S3, S4, S5C, S6, S7, S8C, and S10 is determined to have been performed twice in Yes in Step S10.

Specifically, the energization control portion 323 determines in Step S11 whether or not the temperature difference between the heater temperatures of the first and second resistor patterns 17 and 18, which have been measured, respectively, by performing the processing of the loop of Steps S3, S4, S5C, S6, S7, S8C, and S10 twice, is equal to or greater than a first threshold.

Step S12 is performed in a case where the temperature difference between the heater temperatures of the first and second resistor patterns 17 and 18 has been determined to be equal to or greater than the first threshold in Yes in Step S11.

Specifically, the energization control portion 323, in Step S12, determines, as the predetermined time TC, the energization time for the resistor pattern having a higher heater temperature out of the first and second resistor patterns 17 and 18. Further, the energization control portion 323 sets the energization time for the resistor pattern, which has a lower heater temperature, at a time longer than the predetermined time TC. The energization control portion 323 then stores the respective energization times in the memory 33.

After Step S12, the control device 3 performs the processing of a loop of Steps S3C1 to S9C1, which is similar to the loop of Steps S3 to S9 described in Embodiment 1 described hereinbefore.

Here in Step S8C1, the energization control portion 323 reads from the memory 33 the energization time corresponding to the target heat-generating portion selected in Step S4C 1 out of the respective energization times stored in the memory 33 in Step S12, and in Step S5C1, continually monitors whether or not the energization time has elapsed since the starting of the energization of the target heat-generating portion.

Steps S12 and the loop of Steps S3C1 to S9C1 described hereinbefore correspond to the first control in the disclosed technology.

If the temperature difference between the heater temperatures of the first and second resistor patterns 17 and 18 is determined to be smaller than the first threshold in No in Step S11, the control device 3 performs the processing of a loop of Steps S3C2 to S9C2, which is similar to the loop of Steps S3 to S9 described in Embodiment 1 described hereinbefore.

### Specific Example of Energization Control Method

A description will next be made of a specific example of the energization control method in Embodiment 3.

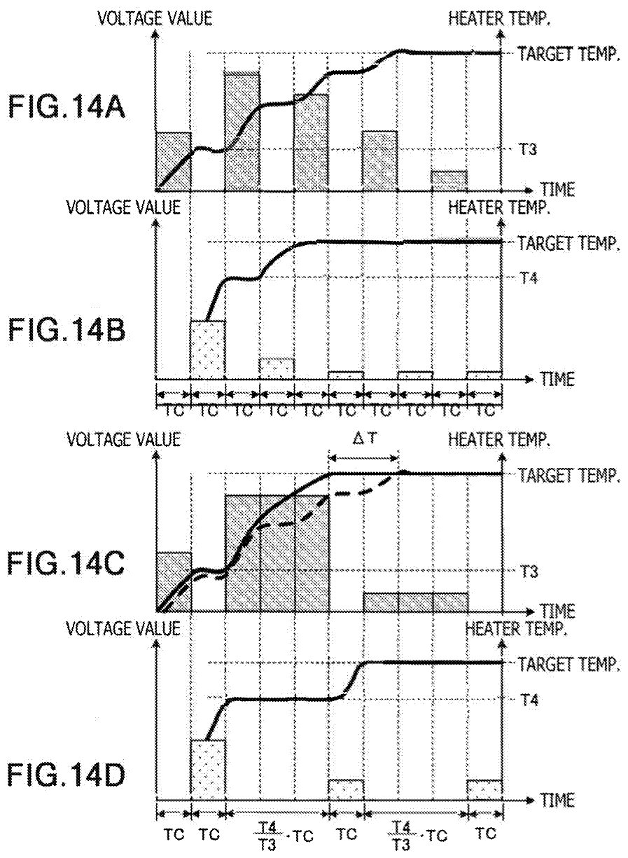

FIGS. 14A-14D indicate graphs illustrating the specific example of the energization control method. Described specifically, FIGS. 14A and 14B are graphs each corresponding to FIGS. 7A-7B, and illustrate changes in heater temperature and voltage value during energization at the first and second resistor patterns 17 and 18, respectively, in a case where energization control is performed by the energization control method described in Embodiment 1 described hereinbefore, specifically the processing of the loop of Steps S3C2 to S9C2 is performed when the temperature difference between the heater temperatures of the first and second resistor patterns 17 and 18 is equal to or greater than the first threshold in Yes in Step 11. FIGS. 14(c) and 14(d) are graphs each corresponding to FIGS. 7A-7B, and illustrate changes in heater temperature and voltage value during energization at the first and second resistor patterns 17 and 18, respectively, in a case where energization control is performed by the energization control method described in Embodiment 3, specifically the processing of Step 12 and the loop of Steps S3C1 to S9C1 is performed when the temperature difference between the heater temperatures of the first and second resistor patterns 17 and 18 is equal to or greater than the first threshold in Yes in Step 11. It is to be noted that FIGS. 14A and 14C illustrate the changes in heater temperature and voltage value during energization at the first resistor pattern 17. In addition, FIGS. 14B and 14D illustrate the changes in heater temperature and voltage value during energization at the second resistor pattern 17. Further, in FIGS. 14A to 14D, the heater temperature of the first resistor pattern 17 and the heater temperature of the second resistor pattern 18 as measured by the processing of the loop of Steps S3, S4, S5C, S6, S7, S8C, and S10 are indicated as a heater temperature T3 and a heater temperature T4, respectively. It is to be noted that the heater temperature T3 is a temperature lower than the heater temperature T4. Further, the temperature difference (T4−T3) between the heater temperatures T3 and T4 is equal to or greater than the first threshold. Therefore, FIGS. 14A and 14B and FIGS. 14C and 14D respectively illustrate cases in which the same temperature difference (T4−T3) arises and the same unevenly distributed load occurs.

In a case where the temperature difference between the heater temperatures of the first and second resistor patterns 17 and 18 is smaller than the first threshold in No in Step 11, the processing of the loop of Steps S3C2 to S9C2 is repeatedly performed, whereby the first and second resistor patterns 17 and 18 are energized with a constant interval, in other words, for every predetermined time TC as in Embodiment 1 described hereinbefore. Accordingly, the heater temperatures of the first and second resistor patterns 17 and 18 are each controlled to the target temperature (see, for example, FIGS. 7A-7B).

In a case where the temperature difference between the heater temperatures of the first and second resistor patterns 17 and 18 is equal to or greater than the first threshold in Yes in Step 11, conversely, the energization time for the second resistor pattern 18 having the higher heater temperature T4 is set at the predetermined time TC in Step S12 as illustrated in FIGS. 14C and 14D. Meanwhile, the energization time for the first resistor pattern 17 having the lower heater temperature T3 is set at a time (T4/T3)·TC calculated by multiplying the predetermined time TC with the ratio of the heater temperature T4 to the heater temperature T3, that is, T4/T3. Then, the processing of the loop of Steps S3C1 to S9C1 is repeatedly performed, and the target heat-generating portion is switched every energization time TC or (T4/T3)·TC, whereby the heater temperatures of the first and second resistor patterns 17 and 18 are each controlled to the target temperature.

According to Embodiment 3 described hereinbefore, the following advantageous effects are brought about in addition to advantageous effects similar to those available from Embodiment 1 described hereinbefore.

It is now assumed that an unevenly distributed load is applied. Between the heater temperatures of the first and second resistor patterns 17 and 18 as measured respectively by performing the processing of the loop of Steps S3, S4, S5C, S6, S7, S8C, and S10 twice, the heater temperature of the resistor pattern covered at a greater region thereof by the biological tissue LT becomes lower than the heater temperature of the other resistor pattern because more heat is transferred from the former resistor pattern to the biological tissue LT.

In the treatment system 1 according to Embodiment 3, with a focus placed on the feature described hereinbefore, the energization time for one of the first and second resistor patterns 17 and 18, the one resistor pattern having the lower heater temperature, is set longer than the energization time for the resistor pattern having the higher heater temperature. In other words, electrical power is positively supplied to one of the first and second resistor patterns 17 and 18, the one resistor pattern being covered at the greater region thereof by the biological tissue LT.

Comparing the case in FIGS. 14A and 14B, in which energization is performed by the energization control method described in Embodiment 1 described hereinbefore, and the case in FIGS. 14C and 14D, in which energization is performed by the energization control method in Embodiment 3, while an unevenly distributed load is applied, the case in which the energization control is performed by the energization control method in Embodiment 3 allows the heater temperature of the resistor pattern, which is covered at the greater region thereof by the biological tissue LT, to reach the target temperature faster. In the case in which the energization control is performed by the energization control method in Embodiment 3, for example, as illustrated in FIG. 14C, the heater temperature of the resistor pattern reaches the target temperature faster by a time ΔT. The treatment time of the biological tissue LT can be shortened accordingly. It is to be noted that the dashed line indicated in FIG. 14C is the same as the solid line indicated in FIG. 14A.

In the treatment system 1 according to Embodiment 3, the first control is performed in Steps S12, and S3C1 to S9C1 in a case where the temperature difference between the heater temperatures of the first and second resistor patterns 17 and 18 is equal to or greater than the first threshold in Yes in Step S11. In other words, the first control is performed only in a case where an unevenly distributed load is pronounced, in other words, in a case where the temperature difference between the heater temperatures of the first and second resistor patterns 17 and 18 is equal to or greater than the first threshold.

In a case where an unevenly distributed load is not pronounced, it is hence unnecessary to perform Step S12, it is possible to reduce the processing load on the control device 3 to the extent that Step S12 is not performed.

### Embodiment 4

A description will next be made about Embodiment 4.

In the following description, similar configurations and steps as in Embodiment 1 described hereinbefore will be identified by the same numeral references, and their detailed description will be omitted or simplified.

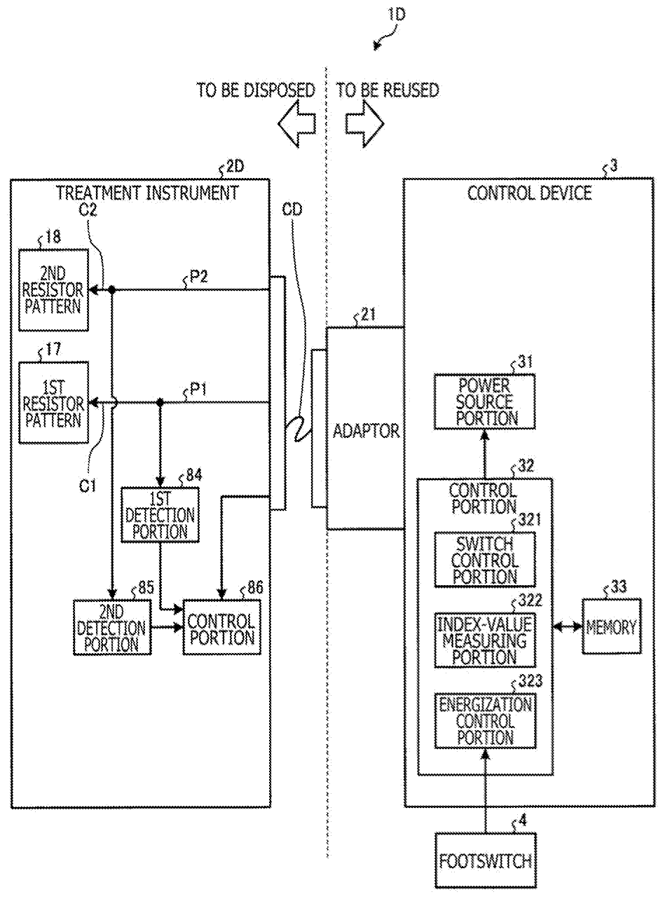

FIG. 15 is a block diagram illustrating a treatment system 1D according to Embodiment 4.

In the treatment system 1 according to Embodiment 1 described hereinbefore, the first and second switch portions 81 and 82 and the switch drive portion 83 are arranged in the treatment instrument 2, for example, in the interior of the handle 5.

In the treatment system 1D according to Embodiment 4, in contrast, a treatment instrument 2D with the first and second switch portions 81 and 82 and the switch drive portion 83 omitted from the treatment instrument 2 is adopted as illustrated in FIG. 15. Also, in the treatment system 1D, an adapter 21 is added detachably from the control device 3. Further, the treatment instrument 2D and the control device 3 are connected to each other via the adapter 21 and an electrical cable CD, so that the control portions 86 and 32 can communicate with each other and electrical power can be supplied from the power source portion 31 to the first and second resistor patterns 17 and 18.

In this case, although a specific illustration is omitted in the figure, the first and second switch portions 81 and 82 and switch drive portion 83 are arranged in the interior of the adapter 21. The treatment instrument 2D and the control device 3 are connected to each other, whereby the first and second switch portions 81 and 82 are disposed in the first and second supply routes P1 and P2, respectively. Also, in Embodiment 4, the switch drive portion 83 is directly controlled by the control portion 32.

According to Embodiment 4 described hereinbefore, the following advantageous effects are brought about in addition to advantageous effects similar to those available from Embodiment 1 described hereinbefore.

In the treatment system 1D according to Embodiment 4, the treatment instrument 2D includes neither the first and second switch portions 81 and 82 nor the switch drive portion 83. Instead, the first and second switch portions 81 and 82 and switch drive portion 83 are arranged in the interior of the adapter 21.

Compared with the treatment instrument 2 described in Embodiment 1 described hereinbefore, it is therefore possible to achieve configurational simplification, dimensional reduction and cost reduction of the treatment instrument 2D. In a case where the treatment instrument 2D is configured as a disposable part to be discarded after use, the first and second switch portions 81 and 82 and switch drive portion 83 can be reused because they are arranged in the adapter 21.

### Other Embodiments

Description has hereinbefore been made of the modes for practicing the disclosed technology. The disclosed technology, however, should not be limited to Embodiments 1 to 4 and Modifications 1 and 2 of Embodiment 1 described hereinbefore.

In Embodiments 1 to 4 and Modifications 1 and 2 of Embodiment 1 described hereinbefore, the second grasping member 10 may be omitted without problem.

Without problem, Embodiments 1 to 4 and Modifications 1 and 2 of Embodiment 1 described hereinbefore may also have a configuration such that an additional heat-generating structure element 12 is included in the second grasping member 10 and thermal energy is applied to the biological tissue LT from both the first and second grasping members 9 and 10.

Without problem, Embodiments 1 to 4 and Modifications 1 and 2 of Embodiment 1 described hereinbefore may also have a configuration such that radio frequency energy or ultrasonic energy may further be applied to the biological tissue LT in addition to thermal energy.

In Embodiments 1 to 4 and Modifications 1 and 2 of Embodiment 1 described hereinbefore, the heat transfer member 13 and the opposing plate 20 are configured as planar surfaces at grasping surfaces thereof, where the heat transfer member 13 and the opposing plate 20 come into contact with the biological tissue LT, but are not limited to such a configuration. For example, the grasping surfaces may also be configured to have a convex, concave, chevron, or like cross-sectional shape.

In Embodiments 1 to 4 and Modifications 1 and 2 of Embodiment 1 described hereinbefore, the energization control of the first and second resistor patterns 17 and 18 is performed based on their heater temperatures measured by the index-value measuring portion 322, but is not limited to such a method. Without problem, the energization control of the first and second resistor patterns 17 and 18 may also be performed, for example, based on the resistance values of the first and second resistor patterns 17 and 18 as measured by the index-value measuring portion 322.

In Embodiments 1 to 4 and Modifications 1 and 2 of Embodiment 1 described hereinbefore, only the two heat-generating portions in the disclosed technology, specifically the first and second main pattern parts 172 and 182 are arranged. Without being limited to this configuration, however, three or more heat-generating portions may also be arranged at positions different in the longitudinal direction of the grasping portion 7 without problem. Further, the number of the switch portions in the disclosed technology is not limited to two (the first and second switch portions 81 and 82), but switch portions may be arranged as many as the heat-generating portions in the disclosed technology or a different number (for example, only one) of heat-generating portion or portions may also be arranged without problem. Furthermore, as the switch portions in the disclosed technology, high-speed mechanical switches or the like may also be used without problem without being limited to FETs.

In Embodiment 3 described hereinbefore, the LEVEL method is adopted for the energization control of the first and second resistor patterns 17 and 18. Without being limited to such a method, the PWM method described in Embodiment 2 described hereinbefore may, however, also be adopted without problem.

In sum, one aspect of the disclosed technology is directed to a treatment system that includes a heat generating structure element having opposed respective distal and proximal ends. The heat generating structure element includes a heat transfer member and a plurality of heat-generating portions coupled to one another. The heat transfer member is configured to transmit thermal energy to a treatment target. The plurality of heat-generating portions is coupled to the heat transfer member along a longitudinal direction extending from the distal end to the proximal end along the heat transfer member so as to transmit heat to the heat transfer member. A power source portion supplies electrical power to the plurality of heat-generating portions. A switch portion selects one target heat-generating portion from the plurality of heat-generating portions to be used as a target to which electrical power is to be supplied from the power source portion. A switch control portion is used to control operation of the switch control portion such that the one target heat-generating portion is sequentially switched from of the plurality of heat-generating portions. An energization control portion is used to control at least one of a switching timing of the target heat-generating portion by the switch control portion and the electrical power to be supplied from the power source portion to the target heat-generating portion.

The treatment system further comprises an index-value measuring portion used to measure respective index values that are to be used as indices of temperatures of the plurality of heat-generating portions. Based on the index values, the energization control portion controls at least one of the switching timing of the target heat-generating portion by the switch control portion and the electrical power to be supplied from the power source portion to the target heat-generating portion. The energization control portion controls the switching timing such that time from stopping a supply of electrical power until starting a next supply of electrical power becomes equal to or smaller than a time constant of temperature changes at the target heat-generating portion. The energization control portion sets the switching timing with a constant interval and controls, a peak value of electrical power to be supplied from the power source portion to the target heat-generating portion. The energization control portion maintains constant a peak value of electrical power to be supplied from the power source portion to the target heat-generating portion and continually energizes the target heat-generating portion to control energization time based on the index value of the target heat-generating portion. The index-value measuring portion measures respective temperatures of the plurality of heat-generating portions and in a condition where one of the plurality of heat-generating portions having a lowest temperature among the plurality of heat-generating portions, is selected as the target heat-generating portion. The energization control portion performs a first control to control at least one of the switching timing and the electrical power to be supplied from the power source to the target heat-generating portion such that electrical power is supplied in a greater quantity to the target heat-generating portion than to each remaining heat-generating portion. The energization control portion performs the first control in a condition where a temperature difference between a lowest temperature and a highest temperature in the plurality of heat-generating portions is equal to or greater than a first threshold. The treatment target is a biological tissue.

Another aspect of the disclosed technology is directed to a treatment system that includes a control device and a treatment instrument configured to be attached to the control device. The treatment instrument includes a handle, a shaft, and a grasping portion for grasping and applying treatment to a treatment target. The grasping portion includes respective first and second grasping members being attached to one another. The first and second grasping members are pivotally supported on one end of the shaft so as to be opened or closed with respect to one another. The first grasping member includes a heat generating structure element having opposed respective distal and proximal ends. The heat generating structure element includes a heat transfer member and a plurality of heat-generating portions coupled to one another. The heat transfer member is configured to transmit thermal energy to the treatment target. The plurality of heat-generating portions is coupled to the heat transfer member along a longitudinal direction extending from the distal end to the proximal end along the heat transfer member so as to transmit heat to the heat transfer member. A power source portion supplies electrical power to the plurality of heat-generating portions. A switch portion selects one target heat-generating portion from the plurality of heat-generating portions to be used as a target to which electrical power is to be supplied from the power source portion. A switch control portion is used to control operation of the switch portion such that the one target heat-generating portion is sequentially switched from of the plurality of heat-generating portions. An energization control portion is used to control at least one of a switching timing of the target heat-generating portion by the switch control portion and the electrical power to be supplied from the power source portion to the target heat-generating portion.

A further aspect of the disclosed technology is directed to a method of operating a treatment system for treatment of a biological tissue. The method comprises transmitting thermal energy to the biological tissue by using a heat generating structure element having opposed respective distal and proximal ends. The heat generating structure element includes a heat transfer member and a plurality of heat-generating portions coupled to one another. Supplying electrical power to the plurality of heat-generating portions via a power source portion. Using a switch portion for selecting one target heat-generating portion from the plurality of heat-generating portions to be used as a target to which electrical power is to be supplied from the power source portion. Controlling operation of the switch portion via a switch control portion such that the one target heat-generating portion is sequentially switched from of the plurality of heat-generating portions, and implementing an energization control portion for controlling at least one of a switching timing of the target heat-generating portion by the switch control portion and wherein electrical power to be supplied from the power source portion to the target heat-generating portion.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A treatment system comprising:
a heat generating structure element having opposed respective distal and proximal ends, the heat generating structure element includes a heat transfer member and a plurality of heat-generating portions coupled to one another, the heat transfer member configured to transmit thermal energy to a treatment target and wherein the plurality of heat-generating portions being coupled to the heat transfer member along a longitudinal direction extending from the distal end to the proximal end along the heat transfer member so as to transmit heat to the heat transfer member,
a power source portion supplying electrical power to the plurality of heat-generating portions,
a switch portion selects one target heat-generating portion from the plurality of heat-generating portions to be used as a target to which electrical power is to be supplied from the power source portion,
a switch control portion being used to control operation of the switch portion such that the one target heat-generating portion is sequentially switched from of the plurality of heat-generating portions, and
an energization control portion being used to control at least one of a switching timing of the target heat-generating portion by the switch control portion and wherein electrical power to be supplied from the power source portion to the target heat-generating portion.

2. The treatment system of claim 1 further comprising:
an index-value measuring portion used to measure respective index values that are to be used as indices of temperatures of the plurality of heat-generating portions,
wherein based on the index values, the energization control portion controls at least one of the switching timing of the target heat-generating portion by the switch control portion and the electrical power to be supplied from the power source portion to the target heat-generating portion.

3. The treatment system of claim 1,
wherein the energization control portion controls the switching timing such that time from stopping a supply of electrical power until starting a next supply of electrical power becomes equal to or smaller than a time constant of temperature changes at the target heat-generating portion.

4. The treatment system of claim 2,
wherein the energization control portion sets the switching timing with a constant interval and controls, a peak value of electrical power to be supplied from the power source portion to the target heat-generating portion.

5. The treatment system of claim 2,
wherein the energization control portion maintains constant a peak value of electrical power to be supplied from the power source portion to the target heat-generating portion and continually energizes the target heat-generating portion to control energization time based on the index value of the target heat-generating portion.

6. The treatment system of claim 2,
wherein the index-value measuring portion measures respective temperatures of the plurality of heat-generating portions and in a condition where one of the plurality of heat-generating portions having a lowest temperature among the plurality of heat-generating portions, is selected as the target heat-generating portion, the energization control portion performs a first control to control at least one of the switching timing and the electrical power to be supplied from the power source to the target heat-generating portion such that electrical power is supplied in a greater quantity to the target heat-generating portion than to each remaining heat-generating portion.

7. The treatment system of claim 6,
wherein the energization control portion performs the first control in a condition where a temperature difference between a lowest temperature and a highest temperature in the plurality of heat-generating portions is equal to or greater than a first threshold.

8. The treatment system of claim of claim 1, wherein the treatment target is a biological tissue.

9. A treatment system comprising:
a control device; and
a treatment instrument configured to be attached to the control device, the treatment instrument includes a handle, a shaft, and a grasping portion for grasping and applying treatment to a treatment target, the grasping portion includes respective first and second grasping members being attached to one another and wherein the first grasping member comprises
a heat generating structure element having opposed respective distal and proximal ends, the heat generating structure element includes a heat transfer member and a plurality of heat-generating portions coupled to one another, the heat transfer member configured to transmit thermal energy to the treatment target and wherein the plurality of heat-generating portions being coupled to the heat transfer member along a longitudinal direction extending from the distal end to the proximal end along the heat transfer member so as to transmit heat to the heat transfer member,
a power source portion supplying electrical power to the plurality of heat-generating portions,
a switch portion selects one target heat-generating portion from the plurality of heat-generating portions to be used as a target to which electrical power is to be supplied from the power source portion,
a switch control portion being used to control operation of the switch control portion such that the one target heat-generating portion is sequentially switched from of the plurality of heat-generating portions, and
an energization control portion being used to control at least one of a switching timing of the target heat-generating portion by the switch control portion and wherein electrical power to be supplied from the power source portion to the target heat-generating portion.

10. The treatment system of claim 9, wherein the first and second grasping members are pivotally supported on one end of the shaft so as to be opened or closed with respect to one another.

11. The treatment system of claim 9 further comprising:
an index-value measuring portion used to measure respective index values that are to be used as indices of temperatures of the plurality of heat-generating portions,
wherein based on the index values, the energization control portion controls at least one of the switching timing of the target heat-generating portion by the switch control portion and the electrical power to be supplied from the power source portion to the target heat-generating portion.

12. A method of operating a treatment system for treatment of a biological tissue, the method comprising:
transmitting thermal energy to the biological tissue by using a heat generating structure element having opposed respective distal and proximal ends, the heat generating structure element includes a heat transfer member and a plurality of heat-generating portions coupled to one another,
supplying electrical power to the plurality of heat-generating portions via a power source portion,
using a switch portion for selecting one target heat-generating portion from the plurality of heat-generating portions to be used as a target to which electrical power is to be supplied from the power source portion,
controlling operation of the switch portion via a switch control portion such that the one target heat-generating portion is sequentially switched from of the plurality of heat-generating portions, and
implementing an energization control portion for controlling at least one of a switching timing of the target heat-generating portion by the switch control portion and wherein electrical power to be supplied from the power source portion to the target heat-generating portion.
